(19) United States
(12) Patent Application Publication
(10) Pub. No.: US 2025/0255714 A1
(43) Pub. Date: Aug. 14, 2025

(54) Vascular Graft And Method Of Fabricating The Same

(71) Applicant: Wisconsin Alumni Research Foundation, Madison, WI (US)

(72) Inventors: Li-Sheng Turng, Madison, WI (US); Edward Chen, Madison, WI (US)

(21) Appl. No.: 18/437,954

(22) Filed: Feb. 9, 2024

(51) Int. Cl. A61F 2/06; A61L 27/18; A61L 27/50; A61L 27/56

(52) U.S. Cl. CPC A61F 2/06; A61L 27/18; A61L 27/507; A61L 27/56; A61F 2240/001

(57) ABSTRACT

A vascular graft and method of fabricating the same are provided. The vascular graft includes an elongated tube fabricated from a material having a plurality of nodes interconnected by a plurality of fibrils. The elongated tube extends along an axis and has been previously stretched along the axis from an initial length to a desired length. Each of the plurality of fibrils has a length, and first and second ends separated by a linear distance. After the second stretching, the length of each of a majority of the plurality of fibrils is greater than the linear distance between the first and second ends of each of the majority of the plurality of fibrils, thereby allowing the vascular graft of the present invention to exhibit higher elastic compliance than prior synthetic, vascular grafts.

### FIELD OF THE INVENTION

The present invention relates generally to vascular bypass procedures conducted for the treatment of severe cardiovascular diseases, and in particular, to a synthetic, vascular graft and method of fabricating the same which has mechanical properties similar to native vascular materials.

### BACKGROUND AND SUMMARY OF THE INVENTION

Polytetrafluoroethylene (PTFE), more widely known as Teflon®, is a polymer material renowned for its unique properties, making it widely employed in industrial applications. This special homopolymer is characterized by its low surface energy, low coefficient of friction, chemical inertness, and biocompatibility. The versatility of PTFE can be seen in many applications, such as additives, gaskets, low-friction materials, and electronics. Stemming from this polymer, a derivative form of PTFE called expanded polytetrafluoroethylene (ePTFE), is produced by mechanically stretching (expansion) of PTFE to create a highly porous structure. First pioneered by Gore, this version of PTFE exhibits increased flexibility and porosity, making it attractive to many applications such as filtration and breathable fabrics as well as medical devices and implants. Further, the processibility and large-scale production of ePTFE have many attractive qualities making it an appealing material of choice in the industry.

One of the fields in which ePTFE is used is in the clinical treatment of severe cardiovascular diseases (CVDs), which have emerged as a leading cause of global mortality. For example, in order to treat CVDs, vascular bypass procedures are widely performed. A vascular procedure is a surgical procedure wherein blood flow is redirected around a diseased artery by means of a vascular graft. Often times, a surgeon will use someone's own vein for the vascular bypass. However, when the availability of suitable autologous conduits is unavailable, prosthetic grafts fabricated from other types of materials must be used. Expanded polytetrafluoroethylene (ePTFE) has demonstrated compatibility as a synthetic substitute for vascular grafts.

Heretofore, ePTFE grafts have achieved commercial success in large-diameter vascular grafts (LDVGs) greater than 6 millimeters (mm). However, in small-diameter vascular grafts (SDVGs) of less than 6 mm, the use of ePTFE grafts for bypass surgeries still has shortcomings. More specifically, SDVGs of less than 6 mm have a patency of approximately 60% after one (1) year. This patency of SDVGs of less than 6 mm falls far short of the patency of autologous grafts, which exhibit a notably higher patency rate of approximately 95%. The viability of synthetic vascular grafts is compromised by various significant factors, including differences in compliance between the graft material and native tissue, hemodynamic considerations at the graft interface, and the inadequacy of sufficient endothelialization. These challenges are further exacerbated in the case of SDVGs, leading to the diminished patency rates for such grafts.

One of the greatest drawbacks of synthetic grafts, and particularly ePTFE vascular grafts, is the mechanical mismatch between the synthetic materials, which lack compliance, and native vascular materials. The mismatch of compliance between synthetic and native vasculature can cause many issues at the interface site, such as thrombosis and intimal hyperplasia. These factors significantly contribute to the failure rate of synthetic grafts, by disrupting the blood flow and creating zones of blood recirculation and low wall shear stresses.

Therefore, it is a primary object and feature of the present invention to provide a vascular graft having a microstructure and mechanical response closer to native vasculature than prior synthetic, vascular grafts.

It is a further object and feature of the present invention to provide a method of fabricating a vascular graft from PTFE which is highly compliant, without the need for supplementary additives or inherent material alterations.

It is a still further object and feature of the present invention to provide a method of fabricating a vascular graft which is simple and inexpensive to manufacture.

In accordance with the present invention, a vascular graft is provided. The vascular graft includes an elongated tube fabricated from a material having a plurality of nodes interconnected by a plurality of fibrils. The elongated tube extends along an axis and has been previously stretched along the axis from an initial length to a desired length. Each of the plurality of fibrils has a length, and first and second ends separated by a linear distance. The length of each of a majority of the plurality of fibrils is greater than the linear distance between the first and second ends of each of the majority of the plurality of fibrils.

It is contemplated for the material to be expanded polytetrafluoroethylene and to have porosity sufficient to allow water vapor to pass therethrough, while limiting the ability of liquid water to pass through the material. It is intended the majority of the plurality of fibrils in the material of the elongated tube to be nonparallel.

In accordance with a further aspect of the present invention, a method of fabricating a vascular graft is provided. The method includes the step of providing an elongated tube extending along an axis. The elongated tube is stretched along the axis to an initial length and subsequently sintered. Thereafter, the elongated tube is stretched along the axis to a final length.

The elongated tube has first and second ends and the method may include the additional step of wrapping the first and second ends of the elongated tube with tape prior to stretching the elongated tube along the axis to the initial length. The elongated tube may be heated prior to stretching the elongated tube along the axis to the initial length, and in addition, the elongated tube may be positioned within an environmental chamber during the stretching the elongated tube along the axis to the in initial length. The environmental chamber is maintained at a desired temperature.

The step of sintering the elongated tube includes the step of rapidly heating the elongated to a selected temperature for a selected time period. After rapidly heating the elongated tube to the selected temperature for the selected time period, the elongated tube is cooled to ambient temperature. Once cooled, the elongated the elongated may be heated prior to stretching the elongated tube along the axis to the final length. Again, the elongated tube is positioned within an environmental chamber maintained at a desired temperature during the stretching of the elongated tube along the axis to the final length. The elongated tube of final length is allowed to rest at ambient temperature after stretching. It is contemplated for the initial length to be equal to the final length and for the elongated tube to be sintered at an intermediate length. The intermediate length is less than the initial length and the final length.

In accordance with a still further aspect of the present invention, a method of fabricating a vascular graft is provided. The method includes the step of providing a specimen of material. The material includes a plurality of nodes interconnected by a plurality of fibrils. The specimen is stretched along an axis to a first extended configuration and sintered. Thereafter, the specimen is stretched along the axis to a second extended configuration and allowed to rest at ambient temperature for a selected time period.

Each of the plurality of fibrils has a length, and first and second ends are separated by a linear distance. The length of each of a majority of the plurality of fibrils is greater than the linear distance between the first and second ends of each of the majority of the plurality of fibrils after the specimen is allowed to rest.

The specimen may be heated prior to stretching the specimen to the first extended configuration and positioned within an environmental chamber maintained at a desired temperature during the stretching to the first extended configuration. The step of sintering the specimen may include rapidly heating the specimen to a selected temperature for a selected time period. After rapidly heating the specimen at the selected temperature for the selected time period, the specimen is cooled to ambient temperature.

The specimen may be heated prior to stretching the specimen to the second extended configuration. Thereafter, the specimen is positioned within an environmental chamber maintained at a desired temperature during the stretching of the specimen to the second extended configuration. It is contemplated for the first and second extended configurations to be the same. In addition, it contemplated to sinter the specimen at an intermediate configuration which is small than the first and second expanded configurations.

### BRIEF DESCRIPTION OF THE DRAWINGS

The drawings furnished herewith illustrate a preferred construction of the present invention in which the above advantages and features are clearly disclosed as well as others which will be readily understood from the following description of the illustrated embodiment.

In the drawings:

FIG. 1 is an isometric view of a synthetic tube for use as a vascular graft in accordance with the present invention;

FIG. 2 is a cross-sectional view of the synthetic tube of the present invention taken along line 2-2 of FIG. 1;

FIG. 3 is a schematic, side elevational view of the synthetic tube of the present invention acting a vascular graft;

FIG. 4 is a schematic, side elevational view of an expansion machine for effectuating an initial step in the fabrication of the synthetic tube of the present invention;

FIG. 5A is a front elevational view of the synthetic tube of the present invention after a first expansion thereof during the initial step depicted in FIG. 4;

FIG. 5B is a front elevational view of the synthetic tube of the present invention after relaxation and an oven for effectuating a further in the fabrication of the synthetic tube of the present invention;

FIG. 6 is an enlarged view showing the nodes and fibrils of the synthetic tube of the present invention during the fabrication process;

FIG. 7 is a schematic, side elevational view of the expansion machine of FIG. 4 effectuating a still further step in the fabrication of the synthetic tube of the present invention;

FIG. 8 is a side elevational view of the synthetic tube of the present invention after a second expansion thereof and an enlarged view showing the nodes and fibrils thereof;

FIG. 9 is an enlarged view showing the nodes and fibrils of the synthetic tube of the present invention;

FIG. 10 is a chart showing various specimens utilized during mechanical test process;

FIG. 11 is a graphical representation of the strain of a first set of specimens at various tensile stresses; and

FIG. 12 is a graphical representation the strain of a second set of specimens at various tensile stresses.

### DETAILED DESCRIPTION OF THE DRAWINGS

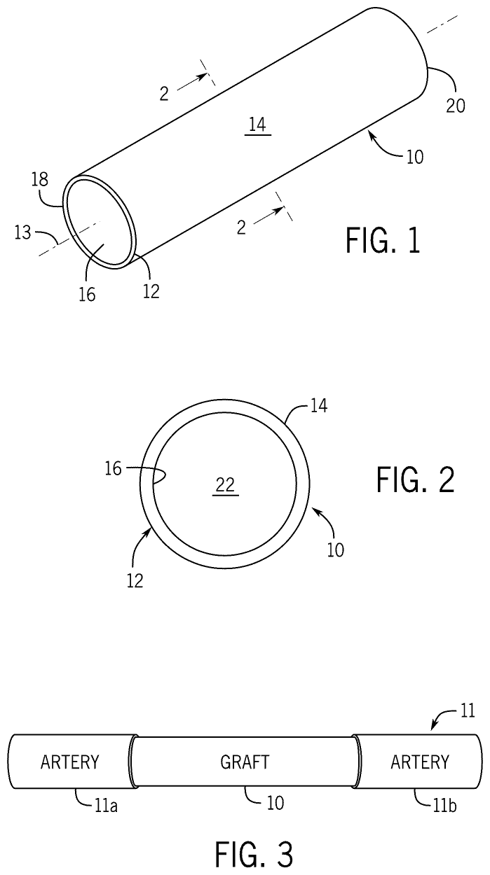

Referring to FIGS. 1-3, a specimen, e.g. a synthetic tube, is generally designated by the reference numeral 10. As best seen in FIG. 2, it is intended for synthetic tube 10 (or a portion thereof) to define a vascular graft for interconnecting first portion 11a of a blood vessel 11 to second portion 11b of blood vessel 11.

Synthetic tube 10 is fabricated from a fluoropolymer, e.g. polytetrafluoroethylene (PTFE). By way of example, synthetic tube 10 may be fabricated from fine PTFE powder particles sieved to produce particles having a diameter in the range from 250 micrometers (μm) to 2000 μm. A lubricant may be provided to facilitate extrusion of the synthetic tube 10, as hereinafter described. A PTFE paste batch, comprising a mixture of 80 grams (g) of PTFE powder particles and 25 g of lubricant, may be mixed in a bottle roller for a selected time period, e.g., in the range of 30 minutes to an hour, at a selected temperature, e.g., less than 19° Celsius (C) to prevent fibrillation among the powder granules and attain even distribution of the PTFE powder particles in the PTFE paste. The resultant mixture is compressed into a preformed cylinder possessing a predetermined diameter, e.g., 50 millimeters (mm) at selected pressure, e.g., a maximum pressure of 2 megapascals (MPa).

The PTFE paste within the cylinder is subjected to extrusion through a die with a desired reduction ratio (RR), e.g., 90:1. The ram velocity may be kept constant, e.g., at 5 mm/min. The extruded PTFE paste defines synthetic tube 10 having an inner diameter of desired size, e.g., 6 mm, and a uniform wall thickness, e.g., 1 mm. Synthetic tube 10 fabricated from the extruded PTFE paste may undergo a vacuum-drying process, e.g., at 40° C., for a minimum duration, e.g., 24 hours, to remove any remnant lubricant from the extruded PTFE paste.

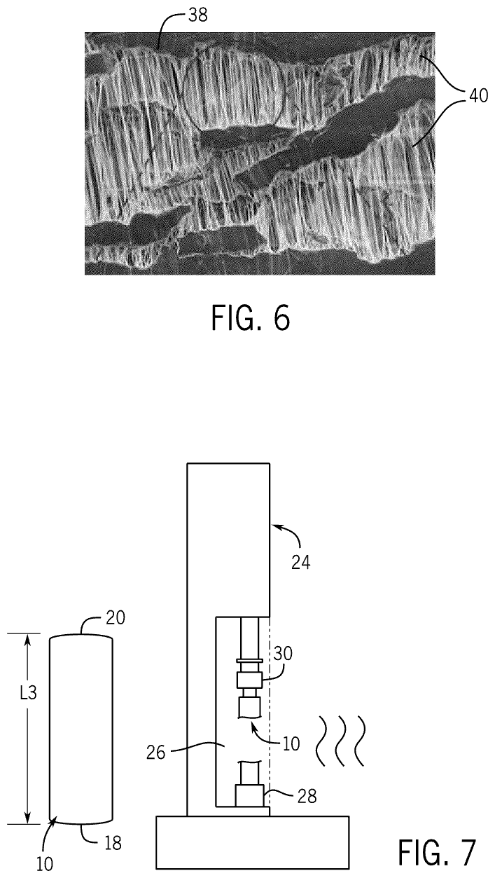

As best seen in FIGS. 1-2, synthetic tube 10 extends along an axis and is defined by a generally cylindrical wall 12 having an outer surface 14, an inner surface 16, first and second ends 18 and 20, respectively. Inner surface 16 of cylindrical wall 12 defines passageway 22 through synthetic tube 10. As is known, the PTFE from which cylindrical wall 12 is fabricated a semi-crystalline polymer defined by a plurality of nodes 38 interconnected by fibrils 40, FIG. 6.

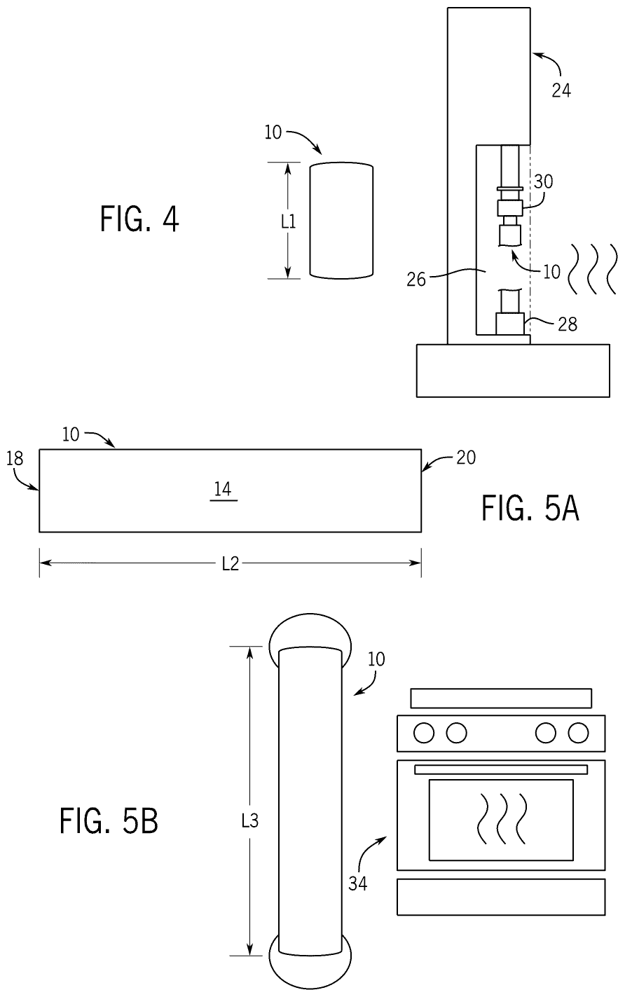

After fabrication, synthetic tube 10 is cut to its initial length L1, e.g., 30 mm. Each of first and second ends 18 and 20, respectively, of cylindrical wall 12 is wrapped, e.g., with ePTFE tape, to prevent cracking during the expansion process, as hereinafter described. Referring to FIG. 4, an expansion machine, e.g., testing machine 24, is provided for stretching synthetic tube 10, as hereinafter described. Testing machine 24 includes environmental chamber 26 to facilitate the heating of synthetic tube 10, as hereinafter described. Synthetic tube 10 is positioned with environmental chamber 26. Thereafter, first end 18 of cylindrical wall 12 is interconnected to base 28 of testing machine 24 in any conventional matter and second end 20 of cylindrical wall 12 is interconnected to crosshead 30 of testing machine 24 in any conventional matter. As hereinafter described, crosshead 30 is vertically movable at a user selected stress level to stretch synthetic tube 10. Synthetic tube 10 is preheated within environmental chamber 26 of testing machine 24 to a selected temperature, e.g., 80° C., for a selected duration, e.g., 5 minutes, to allow synthetic tube 10 to establish a uniform material temperature.

Once synthetic tube 10 establishes a uniform material temperature, environmental chamber 26 is maintained at the selected temperature, e.g., 80° C. and testing machine 24 draws crosshead 30 away from base 28 such that synthetic tube 10 is stretched or expanded under uniaxial tension expansion at a selected rate, e.g., 100 mm/minute, to a first extended length L2 (FIG. 5A), thereby expanding the PTFE material forming cylindrical wall 12 of synthetic tube 10. It can be understood that as cylindrical wall 12 fabricated from PTFE is expanded or stretched, the internodal distance increases, thereby increasing the porosity of cylindrical wall 12. After cylindrical wall 12 of synthetic tube 10 has been expanded, synthetic tube 10 is allowed to relax for a selected relaxation period to minimize the risk of damage to synthetic tube 10 before subsequent manipulation, as hereinafter described. During relaxation, synthetic tube 10 retracts slightly after expansion, e.g., by approximately 10-20 mm, to an intermediate length L3, FIG. 5B.

Following the relaxation period, synthetic tube 10 is fixed at an intermediate length L3 by sintering or subjecting synthetic tube 10 to a rapid heat treatment at a selected temperature, e.g., 360° C., for a selected duration, e.g., 90 seconds. This may be accomplished by transferring synthetic tube 10 from environmental chamber 26 of testing machine 24 into a preheated oven 34 or the like, FIG. 5B. Once synthetic tube 10 has been sintered, synthetic tube 10 is quickly removed from oven 34 to the ambient environment and exposed to flowing air in order to quench synthetic tube to room temperature and prevent the formation of crystalline structures therein. Referring to FIG. 6, it can be seen that after synthetic tube 10 is brought down to ambient/room temperature, the vast majority of fibrils 40 interconnecting nodes 38 in the ePTFE material from which cylinder wall 12 of synthetic tube 10 is formed are generally parallel to each other and generally perpendicular to their corresponding nodes 38.

Referring to FIG. 7, after synthetic tube 10 is brought down to ambient/room temperature, synthetic tube 10 is returned to environmental chamber 26 of testing machine 24. Once again, first end 18 of cylindrical wall 12 is interconnected to base 28 of testing machine 24 in any conventional matter and second end 20 of cylindrical wall 12 is interconnected to crosshead 30 of testing machine 24 in any conventional matter. Synthetic tube 10 is preheated within environmental chamber 26 of testing machine 24 to a selected temperature, e.g., 80° C., for a selected duration, e.g., 5 minutes, to allow synthetic tube 10 to establish a uniform material temperature.

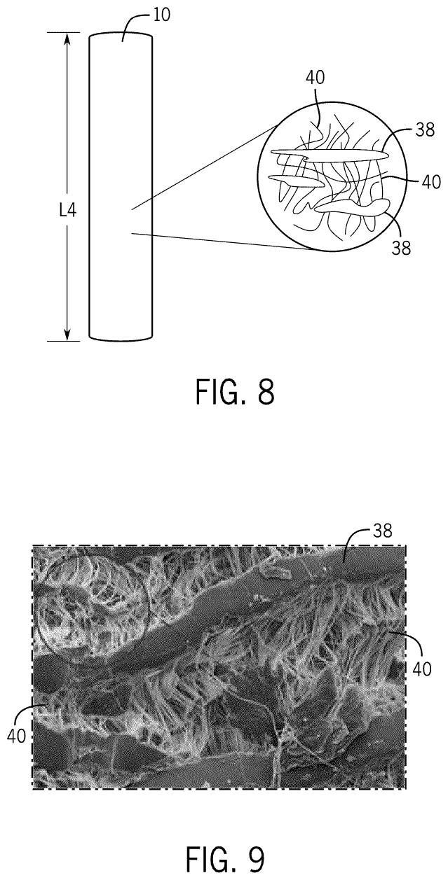

Once synthetic tube 10 establishes a uniform material temperature, with environmental chamber 26 maintained the selected temperature, e.g., 80° C., testing machine 24 draws crosshead 30 away from base 28 such that synthetic tube 10 is stretched or expanded under uniaxial tension expansion at a second selected rate, e.g., 50 mm/minute, to a second expanded length L4, FIG. 8. It is contemplated for the second expanded length L4 to be generally equal to the first expanded length L2 and to be greater than the intermediate length L3. Again, it can be understood that as cylindrical wall 12 is further expanded or stretched, the internodal distance increases, thereby further increasing the porosity of cylindrical wall 12. After cylindrical wall 12 of synthetic tube 10 has been expanded, synthetic tube 10 is removed from environment chamber 26 of testing machine 24 and allowed to relax at ambient/room temperature for a selected relaxation period of at least 24 hours. This ensures the ePTFE material from which synthetic tube 10 fabricated can fully relax to its natural state.

As synthetic tube 10 relaxes, fibrils 40 interconnecting nodes 38 in cylindrical wall 12 of synthetic tube 10 become wavy such that the length of at least a majority of the fibrils 40 is greater than the linear distance between the first and second ends of the fibrils 40, FIG. 9. As hereinafter described, the wavy nature of fibrils 40 contributes to the increased compliance of synthetic tube 10. More specifically, when tensile stress is applied to synthetic tube 10, the wavy fibrils 40 are allowed to extend before becoming engaged in tension. Within the elastic limit of the material, e.g., ePTFE, these wavy fibrils 40 act like springs, thereby allowing the material from which synthetic tube 10 is formed to elastically expand, without permanent deformation. In contrast, in prior synthetic tubes used as vascular grafts, the fibrils in the ePTFE material (after the single-expansion process) do not have a wavy structure, FIG. 6. In other words, the vast majority of the fibrils in prior synthetic tubes used as vascular grafts are generally perpendicular to the nodes and parallel to each other, thereby limiting the elasticity of the material.

It can be appreciated that the introduction of the second expansion step notably altered the morphology of cylindrical wall 12 of synthetic tube 10. This transformation may be attributed to the controlled contraction of certain fibrils 40 during the sintering process, heretofore described. Specifically, allowing some of fibrils 40 to retract facilitated the formation of a disorganized crystal structure, while concurrently, other fibrils 40 amorphously locked into place due to the constrained conditions of the sintering process. Upon re-expanding of synthetic tube 40, the primarily amorphous fibrils 40 undergo plastic deformation and develop residual stress, causing them to partially retract to their sintered length. In contrast, the fibrils 40 that remained structurally locked from the sintering step do not. As such, the different degrees of plastic deformation and elastic contraction among fibrils 40 defining cylindrical wall 12 of synthetic tube 10 causes the fibrils 40 to form a distinctive wavy structure, FIGS. 8-9. As noted above, the wavy structure of fibrils 40 contributes to the increased compliance of synthetic tube 10.

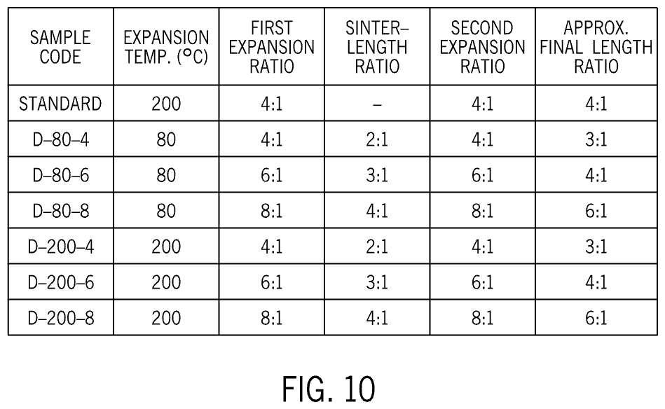

In order to evaluate the mechanical properties of synthetic tube 10, five (5) sets of sample specimens were fabricated and tested. More specifically, each set of sample specimens included a control specimen which underwent a single-expansion and six (6) samples made in accordance with the fabrication process heretofore described (hereinafter after referred to as the “double-expansion” process) were fabricated. Referring to FIG. 10, as hereinafter described, a schematic comparison between the control specimen which underwent a single-expansion and six (6) samples made in accordance with the fabrication process heretofore described is provided.

The synthetic tube 10 designated the control specimen (designated as “Standard” in FIG. 10) was fabricated by preheating the control specimen within environmental chamber 26 of testing machine 24 to 200° C., for a selected duration, e.g., 5 minutes, to allow the control specimen to establish a uniform material temperature. Once the control specimen established a uniform material temperature, with environmental chamber 26 maintained the selected temperature, namely, 200° C., testing machine 24 draws crosshead 30 away from base 28 such that the control specimen was stretched or expanded under uniaxial tension expansion at a selected rate, namely, 500 mm/minute, to a first extended length L2 which had a length increase 4 times the initial length L1 of the control specimen. That is, L2 is five times the initial length L1. Thereafter, the control specimen underwent quenching in a water bath to terminate any relaxation of the fibrils 40.

Each set of sample specimen also included a first set of synthetic tubes 10 that were fabricated in accordance with the double-expansion process heretofore described. A first synthetic tube (designated as “D-80-4” in FIG. 10) of the first set of synthetic tubes 10 was stretched or expanded under uniaxial tension expansion at a rate of 100 mm/minute, to the first extended length L2 which had a length increase 4 times the initial length L1 of synthetic tube 10. After relaxation, the stretched set of synthetic tubes 10 was sintered at intermediate length L3 which had a length increase 2 times the initial length L1. After sintering, the first synthetic tube of the first set of synthetic tubes 10 was stretched or expanded under uniaxial tension expansion at the second selected rate of 50 mm/minute, to second expanded length L4 which had a length increase 4 times the initial length L1. A second synthetic tube (designated as “D-80-6” in FIG. 10) of the first set of synthetic tubes 10 was stretched or expanded under uniaxial tension expansion at a rate of 100 mm/minute, to first extended length L2 which had a length increase 6 times the initial length L1 of synthetic tube 10. After relaxation, the stretched set of synthetic tubes 10 was sintered at intermediate length L3 which had a length increase 3 times the initial length L1. After sintering, the second synthetic tube of the first set of synthetic tubes 10 was stretched or expanded under uniaxial tension expansion at the second selected rate of 50 mm/minute, to second expanded length L4 which had a length increase 6 times the initial length L1. A third synthetic tube (designated as “D-80-8” in FIG. 10) of the first set of synthetic tubes 10 was stretched or expanded under uniaxial tension expansion at a rate of 100 mm/minute, to first extended length L2 which had a length increase 8 times the initial length L1 of synthetic tube 10. After relaxation, the stretched set of synthetic tubes 10 was sintered at intermediate length L3 which had a length increase 4 times the initial length L1. After sintering, the third synthetic tube of the first set of synthetic tubes 10 was stretched or expanded under uniaxial tension expansion at the second selected rate of 50 mm/minute, to second expanded length L4 which had a length increase 8 times the initial length L1.

In addition, each set of sample specimen also included a second set of synthetic tubes 10 (each designated individually as “D-200-4”, “D-200-6” and “D-200-8” in FIG. 10) was similarly formulated, maintaining identical expansion ratios, albeit under an elevated preheating and stretching/expansion temperatures of 200° C. and an increased expansion rate of 500 mm/min. The sintering procedure remained consistent with the first set of synthetic tubes 10.

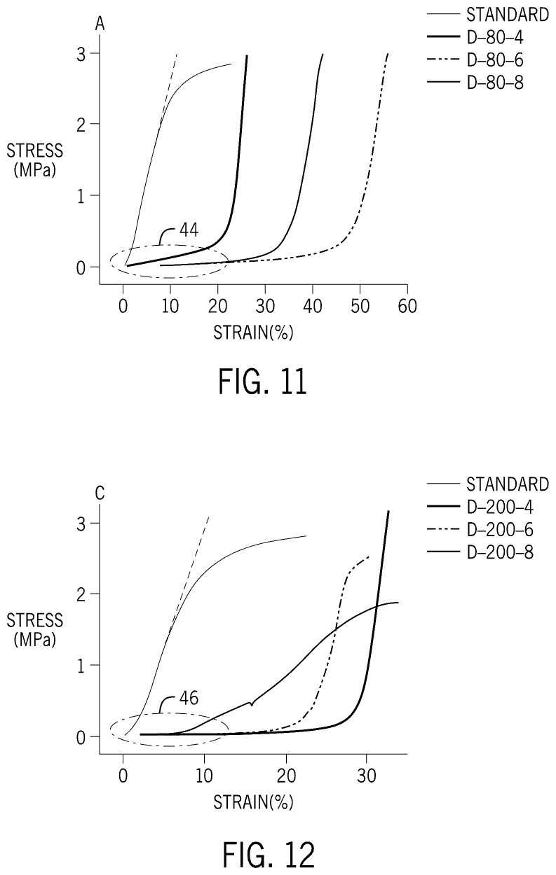

Referring to FIGS. 11-12, to evaluate the mechanical properties of the sample specimens, tensile tests were conducted using a universal testing machine equipped with 30 kilonewton (kN) and 50 newton (N) load cells. Each of the five sets of sample specimens were used to obtain an average of values for each tensile test. For longitudinal tensile tests, each synthetic tube 10 was fixed on both ends and expanded until a maximum stress of 3 MPa was reached or until there was premature failure of synthetic tube 10. Each synthetic tube 10 was stretched at a constant tensile rate of 10 mm/min.

The stress-strain response of the synthetic tubes fabricated in accordance with the double expansion process of the present invention revealed a distinct non-linear behavior consisting of a low-stress/high-strain linear region and a steep, upward linear region, as visually depicted in FIGS. 11-12. The initial low-stress/high-strain region (identified as the toe region 44 in FIG. 11 and toe region 46 in FIG. 12) was a consequence of the wavy configuration of the fibrils 40 being allowed to extend before becoming engaged in tension. Within the elastic limit of the material, e.g., ePTFE, these wavy fibrils 40 act like springs, thereby allowing the material from which synthetic tube 10 is formed to elastically expand, without permanent deformation. The subsequent upward linear region corresponded to the plastic deformation of synthetic tubes 10 occurred when all fibrils 40 were straightened and underwent plastic deformation. In plastic deformation, the synthetic tubes 10 fabricated in accordance with the double expansion process of the present invention behaved relatively closely to the standard, single-expanded control specimen.

In view of the foregoing, it can be appreciated that under normal physiological and vascular conditions, the synthetic tubes 10 fabricated in accordance with the double expansion process of the present invention have drastically greater compliance and strain than the control specimen fabricated in accordance with standard single-expansion process utilized to fabricate current synthetic vascular grafts. Further, it can be understood that by adjusting the process parameters, e.g., the length synthetic tube 10 is stretched prior to and after sintering, in the double expansion process of the present invention, the mechanical properties of synthetic tube 10 may be tuned, without sacrificing the beneficial aspects of utilizing PTFE.

Various modes of carrying out the invention are contemplated as being within the scope of the following claims particularly pointing out and distinctly claiming the subject matter that is regarded as the invention.

We claim:

1. A vascular graft comprising:
an elongated tube fabricated from a material having a plurality of nodes interconnected by a plurality of fibrils and extending along an axis, the tube being previously stretched along the axis from an initial length to a desired length;
wherein:
each of the plurality of fibrils having a length and first and second ends separated by a linear distance; and
the length of each of a majority of the plurality of fibrils is greater than the linear distance between the first and second ends of each of the majority of the plurality of fibrils.

2. The vascular graft of claim 1 wherein the material is expanded polytetrafluoroethylene.

3. The vascular graft of claim 1 wherein the material has a porosity, the porosity being sufficient to allow water vapor to pass therethrough.

4. The vascular graft of claim 3 wherein the porosity of the material limits the ability of liquid water to pass through the material.

5. The vascular graft of claim 1 wherein the majority of the plurality of fibrils are nonparallel.

6. A method of fabricating a vascular graft, comprising the steps of:
providing an elongated tube extending along an axis;
stretching the elongated tube along the axis to an initial length;
sintering the elongated tube; and
stretching the elongated tube along the axis to a final length.

7. The method of claim 6 wherein the elongated tube has first and second ends and wherein the method comprises the additional step of wrapping the first and second ends of the elongated tube with tape prior to stretching the elongated tube along the axis to the initial length.

8. The method of claim 6 comprising the additional step of heating the elongated tube prior to stretching the elongated tube along the axis to the initial length.

9. The method of claim 6 comprising the additional step of positioning the elongated tube within an environmental chamber during the stretching the elongated tube along the axis to the initial length, the environmental chamber being maintained at a desired temperature.

10. The method of claim 6 wherein the step of sintering the elongated tube includes the step of rapidly heating the elongated tube to a selected temperature for a selected time period.

11. The method of claim 10 wherein after the step of rapidly heating the elongated tube to the selected temperature for the selected time period, comprising the additional step of cooling the elongated tube to ambient temperature.

12. The method of claim 6 comprising the additional step of heating the elongated tube prior to stretching the elongated tube along the axis to the final length.

13. The method of claim 12 comprising the additional step of positioning the elongated tube within an environmental chamber during the stretching of the elongated tube along the axis to the final length, the environmental chamber being maintained at a desired temperature.

14. The method of claim 6 comprising the additional step of allowing the elongated tube of final length to rest at ambient temperature after stretching.

15. The method of claim 6 wherein the initial length is the same as the final length.

16. The method of claim 6 wherein the elongated tube is sintered at an intermediate length, the intermediate length being less than the initial length and the final length.

17. A method of fabricating a vascular graft comprising the steps of:
providing a specimen of material, the material including a plurality of nodes interconnected by a plurality of fibrils;
stretching the specimen along an axis to a first extended configuration;
sintering the specimen;
stretching the specimen along the axis to a second extended configuration; and
allowing the specimen to rest at ambient temperature for a selected time period.

18. The method of claim 17 wherein:
each of the plurality of fibrils has a length and first and second ends separated by a linear distance; and
the length of each of a majority of the plurality of fibrils is greater than the linear distance between the first and second ends of each of the majority of the plurality of fibrils after the specimen is allowed to rest.

19. The method of claim 17 comprising the additional steps:
heating the specimen prior to stretching the specimen to the first extended configuration; and
positioning the specimen within an environmental chamber during the stretching to the first extended configuration, the environmental chamber being maintained at a desired temperature.

20. The method of claim 17 wherein the step of sintering the specimen includes the step of rapidly heating the specimen to a selected temperature for a selected time period.

21. The method of claim 20 wherein after the step of rapidly heating the specimen at the selected temperature for the selected time period, comprising the additional step of cooling the specimen to ambient temperature.

22. The method of claim 17 comprising the additional step of heating the specimen prior to stretching the specimen to the second extended configuration.

23. The method of claim 22 comprising the additional step of positioning the specimen within an environmental chamber during the stretching of the specimen to the second extended configuration, the environmental chamber being maintained at a desired temperature.

24. The method of claim 17 wherein the first extended configuration and the second extending configuration are the same.

25. The method of claim 6 wherein the specimen is sintered at an intermediate configuration, the intermediate configuration being smaller than the first extended configuration and the second extended configuration.
